(19) United States
(12) Patent Application Publication
(10) Pub. No.: US 2014/0172051 A1
(43) Pub. Date: Jun. 19, 2014

(54) SINGLE LAYER POLYMER MICROELECTRODE ARRAY

(71) Applicants: Satinderpall S. Pannu, Pleasanton, CA (US); Kedar G. Shah, San Francisco, CA (US); Heeral Sheth, Oakland, CA (US); Sarah H. Felix, Oakland, CA (US); Angela C. Tooker, Dublin, CA (US)

(72) Inventors: Satinderpall S. Pannu, Pleasanton, CA (US); Kedar G. Shah, San Francisco, CA (US); Heeral Sheth, Oakland, CA (US); Sarah H. Felix, Oakland, CA (US); Angela C. Tooker, Dublin, CA (US)

(21) Appl. No.: 13/794,579

(22) Filed: Mar. 11, 2013

Related U.S. Application Data
(60) Provisional application No. 61/737,326, filed on Dec. 14, 2012.

(51) Int. Cl. A61N 1/05

(52) U.S. Cl. CPC A61N 1/0551
USPC 607/116; 264/104

(57) ABSTRACT

A microelectrode array having one or more electrical conduits surrounded and insulated from each other by only a single layer of polymer (e.g. polyimide), and a method of fabricating the same. Multiple layers of an uncured polymer precursor (such as polyamic acid) are separately formed with metal layers sandwiched in between. Formation of the uncured polymer precursor layers includes deposition and heating to remove solvent only but not polymerize the precursor. Upon completing construction, the array is subjected to a high-temperature curing process that converts the uncured polymer precursor layers into the polymer. The different layers of the polymer precursor are thus covalently bonded together during the curing process to create a single continuous layer (e.g. monolithic block) of polymer, with no polymer-polymer interfaces.

### FEDERALLY SPONSORED RESEARCH OR DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### TECHNICAL FIELD

This patent document relates to microelectrode arrays and methods of fabrication, and particularly to a microelectrode array and fabrication method which simultaneously cures multiple uncured polymer precursor layers together to form a single polymer formation surrounding and insulating electrical conduits embedded therein.

### BACKGROUND

Microelectrode neural interfaces are an essential tool in neuroscience, targeting the neuronal activity of neurons, enabling researchers and clinicians to better explore and understand neurological diseases. These interfaces use implanted neural probes to bypass damaged tissue and stimulate neural activity, thereby regaining lost communication and/or control with the affected parts of the nervous system.

Polymer-based microelectrode arrays are widely used. First, they are flexible, thereby minimizing strain between the brain tissue and the implanted array, preventing injury and glial scarring at the implantation site. Second, they are fully biocompatible and thus suitable for chronic implantation with no loss of functionality or safety. Finally, these polymer-based microelectrode arrays can be easily fabricated in large numbers using existing microfabrication techniques.

Traditional microelectrode arrays utilize several layers of metal sandwiched between multiple layers of polymers such as polyimide which is one of the most common polymers used in fabricating polymer-based microelectrode arrays. FIG. 1 shows an example method of forming a polymer-based microelectrode array using multiple layers of polyimide, and shown as a progression of steps 1-11. Step 1, shows a first layer of polyamic acid, labeled A, deposited on a silicon substrate. Next at step 2, a high-temperature curing process is used to convert the polyamic acid into polyimide 1, labeled as B. Next at step 3, a first layer of metal, labeled as C, is deposited and patterned. Next at step 4, a second layer of polyamic acid, labeled as A, is deposited. Next at step 5, the polyamic acid is cured at high temperature to convert the polyamic acid into polyimide 2, labeled as D. Next at step 6, an opening is formed through polyimide 2. Next at step 7, a second layer of metal, labeled as C′, is deposited, including into the opening, and patterned. Next at step 8, a third layer of polyamic acid, labeled as A, is deposited. Next at step 9 the polyamic acid is cured at high temperature to convert the polyamic acid into polyimide 3, labeled as E. Next at step 10, openings are etched through the polyimide 3 (E) to expose the electrodes and produce the device outlines. Next at step 11, the microelectrode array is released from the substrate by separating the polyimide 1 from the substrate.

Unfortunately, the same property that makes polyimide and other polymers useful for biocompatible applications (i.e. its chemical inertness) also makes it difficult to fabricate arrays suitable for long-term implantation into neural tissue. In particular, the adhesion between the various polymer layers is generally quite poor because it relies solely on the weak Van der Waals forces to hold the polyimide layers together. These forces are very weak and can lead to delamination of the polyimide and total failure of microelectrode array device especially when placed in a liquid environment (e.g. in vivo). Therefore, the traditional method for creating polyimide microelectrode arrays which utilizes multiple layers of polyimide (with metal sandwiched in between) as shown in FIG. 1, typically fails quickly when placed in neural tissue. FIGS. 2 and 3 show an example polyimide microelectrode array before and after soaking in a saline solution, respectively. The different layers of polyimide have delaminated (evidenced by the interference fringes in the image) while soaking in a saline solution. This has caused corrosion of the metal sandwiched in between the polyimide layers.

Some work has been done to treat the surface of the cured polyimide in an attempt to convert it back to polyamic acid, as disclosed in the following references: (1) Lee KW, Modification of Polyimide Surface-Morphology—Relationship Between Modification Depth and Adhesion Strength, J. Adhesion Sci. Tech., 8 (10), p. 1077-1092, 1994; (2) Saraf R F, Roldan J M, and Derderian T, Tailoring the Surface-Morphology of Polyimide for Improved Adhesion, IBM J. Res. Devel., 38 (4), p. 441-456, Jul. 1994; and (3) Ranucci E, Sandgren A., Andronova N., and Albertsson A C, Improved polyimide/metal adhesion by chemical modification approaches, J. Appl. Polymer Sci., 82 (8), p. 1971-1985, Nov. 2001. While these methods do improve the adhesion between polyamic acid and cured polyimide, arrays utilizing these treatments are still prone to failure due to delamination between the polyimide layers. In addition, these treatments are not always compatible with the metals used (e.g. titanium) and can etch the metal away completely in the chemistry used to convert cured polyimide to polyamic acid.

### SUMMARY

In one example implementation, a microelectrode array is provided, comprising: an electrical conduit embedded within a simultaneously-polymerized multi-polymer precursor layer-based, single polymer film.

In another example implementation, a method of fabricating a microelectrode array is provided, comprising: forming a multilayer stack having an electrical conduit located between uncured first and second polymer precursor layers which are in contact with each other; and curing the first and second polymer precursor layers together to form a single polymer film surrounding the electrical conduit.

In another example implementation, a method of fabricating a microelectrode array is provided, comprising: forming a multilayer stack having at least two electrical conduits with each electrical conduit arranged between two uncured polymer precursor layers which are in contact with each other; and curing all of the polymer precursor layers together to form a single polymer film surrounding the electrical conduits.

These and other implementations and various features and operations are described in greater detail in the drawings, the description and the claims.

The present invention is generally a microelectrode array having one or more electrical conduits surrounded and insulated from each other by a single layer of polymer, e.g. polyimide, and a method of fabricating the same. Using only one continuous layer of polymer eliminates the various polymer-polymer interfaces in traditional microelectrode arrays and prevents device failure due to polymer delamination. Thus, the lifetime of the device is greatly extended and is more suitable for long-term implantation into, for example, neural tissue when used as a neural interface for either acute or chronic studies of various neurological disorders (e.g. clinical depression, Parkinson's disease, epilepsy) and as interfaces between neural tissue and prosthetics (e.g. retinal implants, auditory implants).

The single-film polymer microelectrode array of the present invention utilizes multiple layers of an uncured polymer precursor (e.g. polyamic acid for producing polyimide) with metal layers sandwiched in between. In particular, the uncured polymer precursors are formed by depositing a polymer precursor solution which is then heated to remove solvent but not polymerize the polymer precursor. Once the fabrication of the array is complete, the device is subjected to a high-temperature curing process that converts the uncured polymer precursor into polymer. The different layers of polymer precursor are thus covalently bonded together during the curing process. This creates a single continuous layer (e.g. monolithic block) of polymer with no polymer-polymer interfaces (i.e. the array does not rely on the weak Van der Waals forces to prevent polymer delamination). Further, this array fabrication does not utilize any adhesion treatments that may adversely affect the metal.

### BRIEF DESCRIPTION OF THE DRAWINGS

FIG. 1 is an example method of fabricating microelectrode arrays known in the prior art.

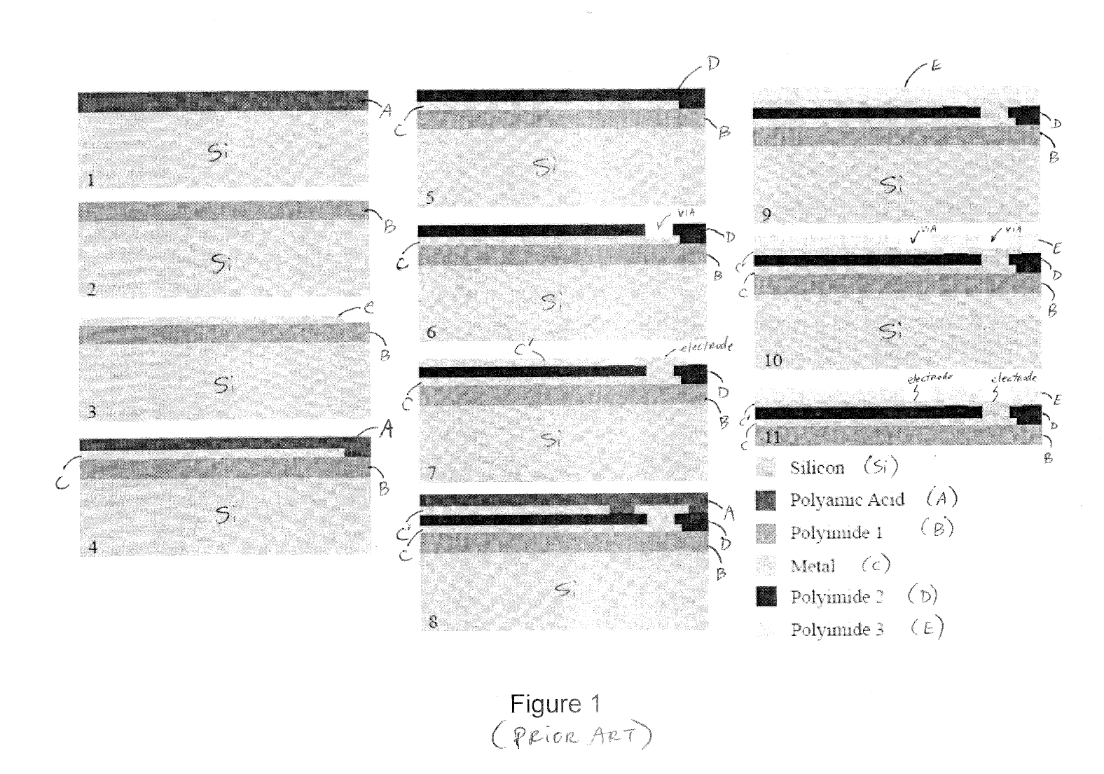

FIGS. 2 and 3 are photos of a microelectrode array before and after, respectively, soaking the microelectrode array in a saline solution, illustrating delamination of polyimide layers causing device failure.

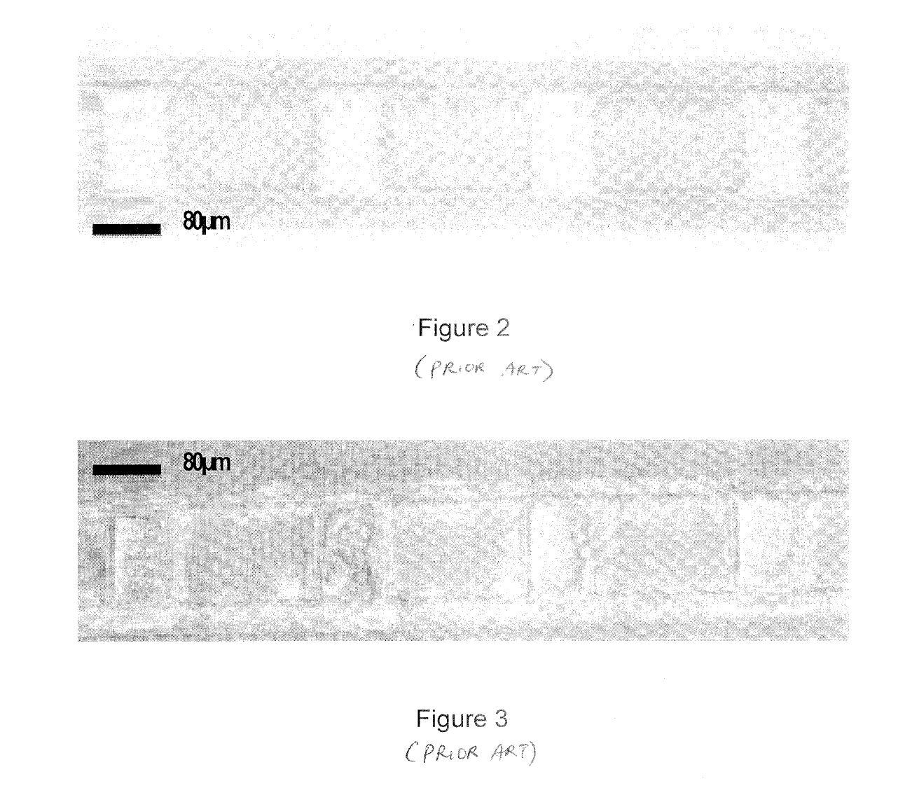

FIG. 4 is an example method of fabricating the microelectrode array of the present invention.

### DETAILED DESCRIPTION

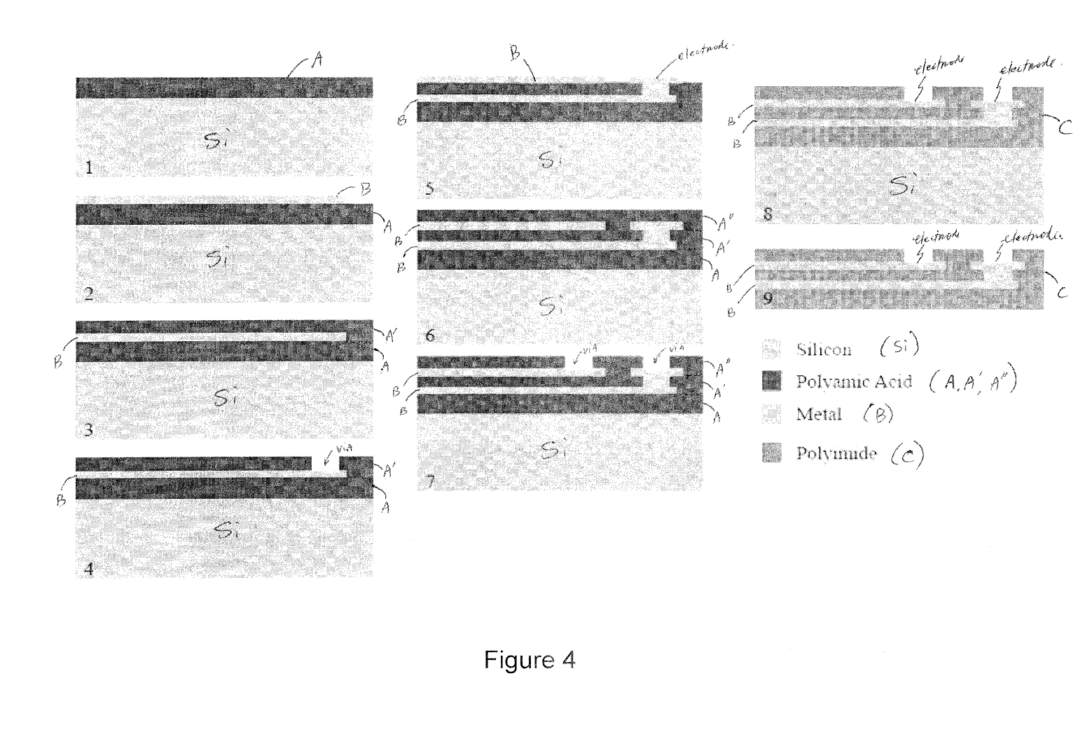

Turning now to the drawings, FIG. 4 shows an example embodiment of the fabrication process of the present invention. In this embodiment, the exposed portions of the electrical conduits (which may be used as electrodes or further connected with bond pads), are described as being formed face up, i.e. exposed from the top. It is appreciated however that in the alternative, the exposed portions may be formed facing down.

Step 1, shows a first layer of uncured polymer precursor, labeled A, formed on a silicon substrate. In particular, the uncured polymer precursor is produced by depositing a polymer precursor solution on the substrate, followed by heating the solution at a relatively low temperature to remove solvent therefrom but not polymerize the polymer precursor (e.g. to about 100 degrees C. for evaporating solvent form polyamic acid solution). It is appreciated that various types of polymer precursors may be utilized, especially those types held in solvent and cured by a high temperature curing process, for producing thermosetting polymers such as for example polyesters, polyurethanes, polyimides, silicones, parylenes, etc., but the present invention is not limited only to such. For example, polyamic acid is one known type of polymer precursor held and solvent and cured by heating to form polyimide. It is appreciated however that polyimide is made up of at least two precursors, one with an imide group and another with a carboxylic acid group, but can be any number of functional groups. Also silicone precursors, such as vinyl groups, vinyl ethers, epoxy groups, ethyl vinyl ethers, acetates, carbolic acids, etc. may also be used. And parylene is another alternative polymer which may be used. And it is appreciated that silicon is one type of material which may be used as the substrate material, and other materials may be used provided that it is compatible with the techniques and chemicals used during the microfabrication.

At step 2, a first electrical conduit, labeled as B, is then formed on the uncured first polymer precursor layer, for example, by depositing a layer of metal and patterning the metal layer into the electrical conduit. Various types of electrically conductive materials may be used for the conduits, such as for example, gold, platinum, iridium, rhodium, titanium, tantalum, ruthenium, niobium, and other noble or inert metals, but is not limited only to such. And various deposition methods may also be used, as known in the art, such as for example, sputtering, thermal or electron-beam evaporation, etc, but is also not limited only to such. It can also be various layers of different metals to form the electrical circuit. Also various patterning methods may be employed, such as for example photolithography, stepper photolithography, e-beam photolithography, stereolithography, direct patterning, direct ink-writing, stencil printing, shadow masking, which may also be combined with wet chemical etching or dry chemical etching such as reactive-ion etching, ion milling, or sputter etching. And the conductive layer may also me patterned in a “grid” or “mesh” pattern in order to allow the solvent to escape during the heating step. Furthermore, the top surface of the bottom uncured polymer precursor layer may be modified with a plasma (such as an oxygen plasma) to “erase” any surface modifications from the metal deposition or patterning steps. Or in the alternative, can also use wet chemical methods to modify the surface of the polymer to remove any modification from the uncured polymer. The microelectrode array may also be fabricated without exposing it to any “wet chemicals”, such as by patterning the electrically conductive layer using a “shadow mask,” instead of depositing everywhere and removing the un-necessary regions.

At step 3, a second layer of polymer precursor, labeled as A′, is formed on the first electrical conduit and the uncured first polymer precursor layer. Similar to step 1, the second polymer precursor layer is produced by depositing a second polymer precursor solution, followed by heating the solution to remove solvent therefrom but not polymerize the polymer precursor.

Then at step 4, an opening is shown formed through the second polymer precursor layer to expose a portion of the underlying metal layer. And at step 5, a second electrical conduit B is formed, similar to that described for step 2. As can be seen at step 5, the formation of the second electrical conduit is by depositing an electrically conductive layer and patterning the electrically conductive layer to form an electrical via that is electrically connected to the first electrical conduit, and electrically insulated from the second electrical conduit.

At step 6, a third layer of polymer precursor, labeled as A″, is formed. Similar to steps 1 and 3, the third polymer precursor layer is produced by depositing a third polymer precursor solution on the second electrical conduit and the second polymer precursor layer, followed by heating the solution to remove solvent therefrom but not polymerize the polymer precursor. It is appreciated that additional layers of metal may be produce by repeating steps 4-6 as many times as necessary. If only a single layer of metal is required, steps 4-6 can be eliminated.

Then at step 7, openings are etched to expose a portion of the underlying second electrical conduit and the electrical via connected to the first electrical conduit. The exposed portions may be used as electrodes or for further fabrication of a bond pad region that is used to connect to electronics or a connector. It is also appreciated that openings for exposing any or all of the electrical conduits maybe be created before or after the curing step (described next).

Next at step 8 all layers of the polymer precursor are cured at high temperature to convert the polymer precursor into a polymer such as polyimide, labeled as C. The temperature range for curing (i.e. imidizing the polymer precursor for polyimide), is typically above 60° C. For example, polyamic acid is imidized and cured at 180° C. to 400° C. Furthermore, the substrate may be adapted to accommodate the release of polymerization reaction by-products produced from the curing step so as to prevent damage to the microelectrode array. For example, a substrate that is porous to the gaseous by-products may be selected which allows the by-products to diffuse from both the top and bottom of the microelectrode array. In the alternative, portions of the substrate, e.g. the backside of the substrate, can be fully or partially removed to allow reaction by-products (e.g. condensation) to diffuse through both the front and back of the microelectrode array and prevent bubbles or other defects in the polymer films. In particular, most of the substrate may be removed from underneath the device while supporting a perimeter of the device. In another alternative, the substrate can be patterned to further allow the reaction by-products to be released without damaging the polymer films in the microelectrode array. For example pockets, voids, or pores may be formed on the surface of the substrate in which to receive the release of by-products. In particular, the pockets may be patterned as weep channels for channeling by-products out from the substrate.

At step 9, the microelectrode array is released from the substrate by separating the polymer (e.g. polyimide) from the substrate. It is appreciated that the releasing step may be performed before or after the curing step. In particular, since polymerization reactions are typically condensation reactions (i.e. they liberate water) and the water has to get out of the polymer, releasing the uncured polymer precursor from the substrate prior to curing would enable water to leave the precursor from the top and bottom. Also, the release step may be performed by, for example, (1) the use of and chemically etching away a sacrificial layer, (2) mechanically releasing, e.g. peeling, or (3) the use of and electrochemically etching of a sacrificial layer. In some cases, a metal release layer (e.g. chrome, titanium, gold) is deposited on the starting silicon substrate prior to the first step of the fabrication process to ensure an easy release of the final device.

Although the description above contains many details and specifics, these should not be construed as limiting the scope of the invention but as merely providing illustrations of some of the presently preferred embodiments of this invention. Other implementations, enhancements and variations can be made based on what is described and illustrated in this patent document. The features of the embodiments described herein may be combined in all possible combinations of methods, apparatus, modules, systems, and computer program products. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Therefore, it will be appreciated that the scope of the present invention fully encompasses other embodiments which may become obvious to those skilled in the art. In the claims, reference to an element in the singular is not intended to mean “one and only one” unless explicitly so stated, but rather “one or more.” All structural and functional equivalents to the elements of the above-described preferred embodiment that are known to those of ordinary skill in the art are expressly incorporated herein by reference and are intended to be encompassed by the present claims. Moreover, it is not necessary for a device to address each and every problem sought to be solved by the present invention, for it to be encompassed by the present claims. Furthermore, no element or component in the present disclosure is intended to be dedicated to the public regardless of whether the element or component is explicitly recited in the claims. No claim element herein is to be construed under the provisions of 35 U.S.C. 112, sixth paragraph, unless the element is expressly recited using the phrase “means for.”

We claim:

1. A microelectrode array, comprising:
an electrical conduit embedded within a simultaneously-polymerized multi-polymer precursor layer-based, single polymer film.

2. The microelectrode array of claim 1,
wherein the polymer is selected from the group consisting of polyimide, parylene, silicone, polyurethane, polyester, polystyrene, polytetrafluoroethylene (PTFE) and polyanhydrides.

3. The microelectrode array of claim 1,
wherein a portion of said conduit is exposed through the single polymer film.

4. The microelectrode array of claim 1,
further comprising at least one additional electrical conduit embedded within the single polymer film and separated from an adjacent electrical conduit by a region of the single polymer film corresponding to one of the simultaneously-polymerized, polymer precursor layers from which the single polymer film was formed.

5. The microelectrode array of claim 4,
wherein each additional conduit has a portion exposed through the single polymer film.

6. A method of fabricating a microelectrode array, comprising:
forming a multilayer stack having an electrical conduit located between uncured first and second polymer precursor layers which are in contact with each other; and
curing the first and second polymer precursor layers together to form a single polymer film surrounding the electrical conduit.

7. The method of claim 6, wherein the multilayer stack is formed by:
depositing a first polymer precursor solution on a substrate;
heating the first polymer precursor solution to remove solvent therefrom but not polymerize the polymer precursor, to form the uncured first polymer precursor layer;
forming an electrical conduit on the first polymer precursor layer;
depositing a second polymer precursor solution on the first polymer precursor layer and the electrical conduit; and
heating the second polymer precursor solution to remove solvent therefrom but not polymerize the polymer precursor, to form the uncured second polymer precursor layer in contact with the first polymer precursor layer and the electrical conduit.

8. The method of claim 6,
wherein the polymer is selected from the group consisting of polyimide, parylene, silicone, polyurethane, polyester, polystyrene, polytetrafluoroethylene (PTFE) and polyanhydrides.

9. The method of claim 6,
further comprising forming an opening through the second polymer precursor layer to expose a portion of the electrical conduit.

10. The method of claim 6,
wherein the electrical conduit is formed by depositing an electrically conductive layer and patterning the electrically conductive layer into the electrical conduit.

11. The method of claim 6,
wherein the multilayer stack is formed on a substrate, and further comprising releasing the single polymer film from the substrate.

12. The method of claim 11,
wherein the single polymer film is released from the substrate before curing.

13. The method of claim 6,
wherein the multilayer stack is formed on a substrate adapted to accommodate the release of polymerization reaction by-products from the curing step and thereby prevent damage to the microelectrode array.

14. The method of claim 13,
wherein the substrate has pockets formed in which to receive the release of polymerization reaction by-products.

15. The method of claim 14,
wherein the pockets are weep channels for channeling by-products out from the substrate.

16. A method of fabricating a microelectrode array, comprising:
forming a multilayer stack having at least two electrical conduits with each electrical conduit arranged between two uncured polymer precursor layers which are in contact with each other; and
curing all of the polymer precursor layers together to form a single polymer film surrounding the electrical conduits.

17. The method of claim 16, wherein the multilayer stack is formed by:
depositing a first polymer precursor solution on a substrate;
heating the first polymer precursor solution to remove solvent therefrom but not polymerize the polymer precursor, to form an uncured first polymer precursor layer;
forming a first electrical conduit on the first polymer precursor layer;
depositing a second polymer precursor solution on the first polymer precursor layer and the first electrical conduit;
heating the second polymer precursor solution to remove solvent therefrom but not polymerize the polymer precursor, to form an uncured second polymer precursor layer in contact with the first polymer precursor layer and the first electrical conduit;
forming an opening through the second polymer precursor layer to expose a portion of the first electrical conduit;
depositing an electrically conductive layer on the second polymer precursor layer and the exposed portion of the first electrical conduit;
patterning the electrically conductive layer to form an electrical via that is electrically connected to the first electrical conduit, and a second electrical conduit electrically insulated from the first electrode;
depositing a third polymer precursor solution on the second polymer precursor layer, the first electrode, and the second electrical conduit; and
heating the third polymer precursor solution to remove solvent therefrom but not polymerize the polymer precursor, to form an uncured third polymer precursor layer in contact with the second polymer precursor layer, the first electrode, and the second electrical conduit; and
forming openings through the second polymer precursor layer to expose the electrical via and a portion of the second electrical conduit.

18. The method of claim 16,
wherein the polymer is selected from the group consisting of polyimide, parylene, silicone, polyurethane, polyester, polystyrene, polytetrafluoroethylene (PTFE) and polyanhydrides.

19. Thefs method of claim 16,
further comprising forming openings through the polymer precursor layers to expose portions of the electrical conduits.

20. The method of claim 16,
wherein the electrical conduits are each formed by depositing an electrically conductive layer and patterning the electrically conductive layer into the electrical conduit.

21. The method of claim 16,
wherein the multilayer stack is formed on a substrate, and further comprising releasing the single polymer film from the substrate.

22. The method of claim 21,
wherein the single polymer film is released from the substrate before curing.

23. The method of claim 16,
wherein the multilayer stack is formed on a substrate adapted to accommodate the release of polymerization reaction by-products from the curing step and thereby prevent damage to the microelectrode array.

24. The method of claim 23,
wherein the substrate has pockets formed in which to receive the release of polymerization reaction by-products.

25. The method of claim 24,
wherein the pockets are weep channels for channeling by-products out from the substrate.
